(19) United States
(12) Patent Application Publication
(10) Pub. No.: US 2006/0252989 A1
(43) Pub. Date: Nov. 9, 2006

(54) Endoscopic apparatus provided with inflatable propelling sleeve

(75) Inventors: Yakov Bar-Or, Haifa (IL); Amram Aizenfeld, Kibbutz Ramot Menashe (IL); Golan Salman, Tirat Hacarmel (IL)

Correspondence Address: BRUCE E. LILLING;LILLING & LILLING P.C., P.O. BOX 560, GOLDEN BRIDGE, NY, 10526, US

(73) Assignee: Sightline Technologies Ltd., Haifa (IL)

(21) Appl. No.: 11/121,691

(22) Filed: May 4, 2005

(51) Int. Cl. A61B 1/04; A61B 1/00

(52) U.S. Cl. 600/115; 600/114; 600/116

(57) ABSTRACT

An apparatus for endoscopic examination of a body passage includes a flexible endoscope and an inflatable sleeve, which upon inflation is capable of propelling the endoscope within the body passage. The endoscope is provided with a proximal end and with a distal end, which comprises an insertion tube, a bending section and a probe. The sleeve comprises a rear folded portion and a frontal unfolded portion. The sleeve is detachably coupled with the insertion tube, wherein the rear portion of the sleeve is located behind the bending section. During the endoscopic examination the rear portion unfolds upon inflation and propels the endoscope within the body passage, while the frontal portion remains deflated and covers the bending section and the probe.

### FIELD OF THE INVENTION

The present invention relates generally to the field of endoscopy and specifically to endoscopes used for colonoscopic procedure during which a flexible tube is inserted into the rectum and colon for examining the colon interior for abnormalities. More particularly, the present invention refers to such an endoscope, which is provided with disposable sleeve covering the tube and which, upon inflation, assists advancement of the colonoscope within the colon.

### BACKGROUND OF THE INVENTION

There are known endoscopes employing inflatable flexible sleeves for propulsion of an endoscope within the colon.

Voloshin (U.S. Pat. No. 6,485,409) discloses an endoscope, which comprises an endoscopic probe, a bending section for directing the probe within the colon (steering unit), an insertion tube and a flexible covering sleeve or a sheath, which is coupled proximally to the probe. The bending section of the endoscope is located behind the probe. The sleeve is attached to the endoscope in such a manner that its folded section is retained between a cap and an internal spindle, which are located between the insertion tube and the probe head. When inflated, the folded section unfolds over a flange of the internal spindle and an inner portion of the sleeve is pulled in a distal direction. Since the sleeve covers merely the insertion tube and neither the steering unit nor the probe, these portions of the endoscope can be contaminated during the colonoscopic procedure and therefore the entire endoscope should be disinfected before reuse. No disclosure is provided as to how the sleeve is anchored to the cap or to the insertion tube.

In PCT/IL03/00661 an endoscope is described which employs a flexible inflatable sleeve, which before inflation is retained within a dispenser. To protect the endoscope from contamination, the sleeve is put thereon in such a manner that it covers the insertion tube, bending section and the probe. Unfortunately this measure reduces maneuverability of the bending section, since upon inflation the sleeve renders some rigidity to the bending section. Accordingly, advancing the endoscope within the colon becomes more difficult and operation of the endoscope is less convenient.

### SUMMARY OF THE INVENTION

The object of the present invention is to provide a new and improved endoscope employing an inflatable sleeve for its propulsion within the colon. The endoscope of the present invention is fully protected from contamination, since the inflatable, disposable sleeve covers the insertion tube, the bending section and the probe. At the same time, by virtue of locating the folded section of the sleeve behind the bending section, maneuverability of the bending section is not compromised. For a better understanding of the present invention as well of its benefits and advantages, reference will now be made to the following description of its embodiments, taken in combination with the accompanying drawings.

### BRIEF DESCRIPTION OF THE DRAWINGS

FIG. 1 depicts a general view of an endoscopic apparatus, preferably a colonoscopic apparatus of the present invention and its main components

FIGS. 2a and 2b are longitudinal views and show schematically how the endoscope of the present invention advances within the colon by being propelled by the inflating sleeve.

FIG. 3 is a longitudinal view, partially broken away, and shows the anchoring and sealing arrangement for attaching the sleeve to the insertion tube.

FIG. 4 in an enlarged view, partially in section and partially broken away, and shows an embodiment of the anchoring and sealing arrangement.

### DETAILED DESCRIPTION OF THE INVENTION

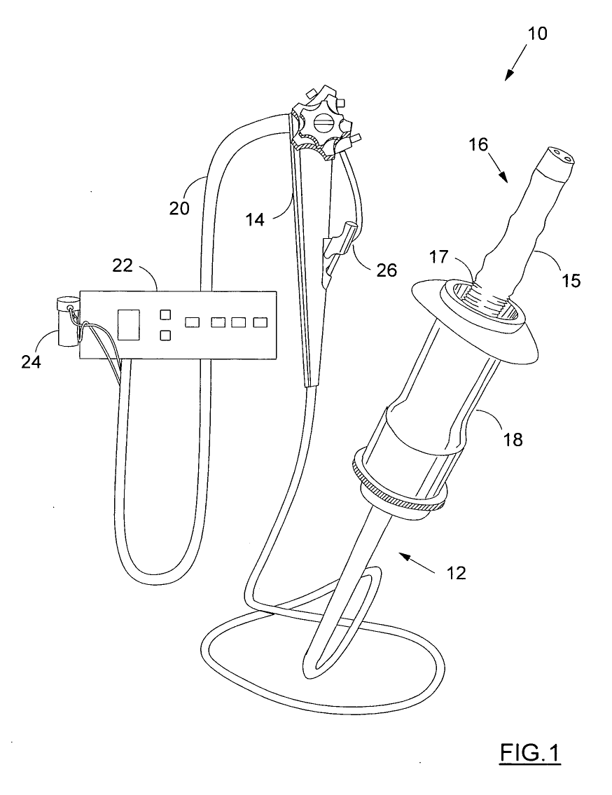

With reference to FIG. 1 a colonoscopic apparatus 10 of the present invention is shown with its following main components. The apparatus comprises an endoscope having an insertion tube with its proximal section 12 connected to an operation handle 14 and with its distal section 16 inserted in and protruding from a disposable dispenser 18. An example of a suitable dispenser and explanation of its functioning can be found in the above referred to PCT/IL03/00661.

One should bear in mind that the distal section of the endoscope is provided with a bending section for maneuvering the endoscope and with a probe, which terminates at its forwardmost end by an appropriate optical head enabling visualization of the colon's interior. It is seen also in FIG. 1 that a sleeve covers the protruding distal section of the endoscope. The distal part of the sleeve, which is seen in FIG. 1, comprises a frontal non-inflatable portion 17 and a rear folded portion 15. It will be explained further in more details that the frontal portion covers the bending section of the endoscope and its optical head. The frontal portion of the sleeve does not inflate when the endoscope advances within the colon. The rear portion covers the insertion tube and unfolds when air or another fluid medium is supplied to the sleeve. By virtue of this provision the endoscope is propelled by the sleeve within the colon. Explanation of this phenomenon can be found in the above referred-to references. The endoscope of the present invention is of similar type in the sense that it employs the same propelling mechanism, which is based on inflation of a flexible sleeve coupled to the endoscope's distal section. The endoscope of the invention is of the similar type also in the sense that it employs a disposable sleeve, which protects the endoscope from contamination.

It should be emphasized, however, that the endoscope of the present invention is not limited merely to colonoscopy. It can be employed in any other medical procedure requiring insertion of a probe in a body passage for inspection of its interior.

It is seen also in FIG. 1, that the operation handle is connected by an appropriate umbilical duct 20 to a control unit 22, which is provided with a source of compressed air for inflating and venting the sleeve. It is not shown specifically, but should be appreciated, that the control unit comprises appropriate solenoids for activating pinch valves for the air, water and suction tubes passing along the umbilical duct. Near the control unit 20, there is provided a flask 24 filled with water, which can be supplied under pressure into the colon for irrigation.

It is not shown in detail, but one should bear in mind that within the insertion tube are provided various devices, which are necessary for proper functioning of the endoscope. These devices are known in the art. Among such devices one can mention vertebrae and strings, which can be manipulated by the handle, and a working channel, or so-called multilumen, with passages for supplying water for irrigation or a vacuum for suction. The multilumen is provided also with a passage for introducing surgical instruments into the colon as might be required during the endoscopic procedure. The multilumen extends through the endoscope and through the handle to a connector means 26, which connects it to the control unit and to the flask.

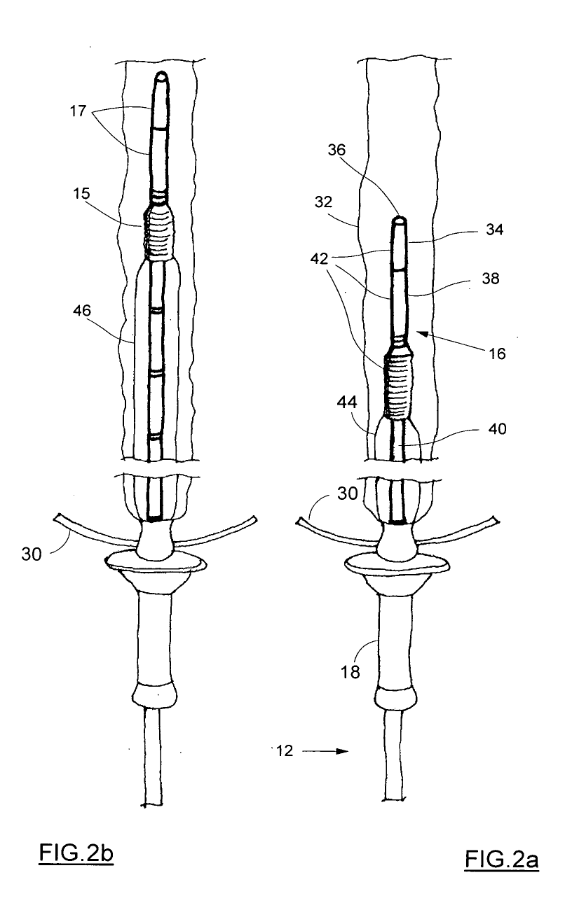

In FIG. 2a there is shown the distal section of the endoscope being inserted into a body passage of a patient, e.g. the colon. The disposable dispenser 18 and the proximal section 12 of the endoscope remain outside of the body passage. A dedicated garment 30 can support the rearmost part of the distal section, for example as explained in International patent application PCT/IL2004/000372. By virtue of this provision more convenient operation of the endoscope is possible.

Referring now to FIGS. 2a, 2b it is seen that distal section 16 of the endoscope extends along the colon 32. The distal section comprises a probe section 34 provided with a camera head 36 fitted with illumination means and viewing optics as required for visualization of the colon's interior (not shown). The distal section also comprises a bending section 38, which is situated behind the probe section. Furthermore the distal section includes the forwardmost region of an insertion tube 40.

A flexible sleeve 42 surrounds entire the length of the distal section. The sleeve is manufactured of a flexible biocompatible plastic material, such as polyamide having a thickness of preferably about 20 microns. It is seen that the frontal non-inflatable portion 17 of the sleeve covers both the bending section and the probe, while the rear folded portion 15 of the sleeve covers merely the forwardmost region of the insertion tube. The folded portion of the sleeve is arranged as a concertina and, when pressure is supplied to the sleeve, the folded portion begins to unfold and reduce its length on account of the unfolded inflated region, which is designated by reference numeral 44. In FIG. 2b is shown how the endoscope advances within the body passage by being propelled when the sleeve inflates. It is seen that inflated region increases its length, as designated by reference numeral 46.

One should bear in mind, however, that since the folded portion is located behind the bending section, the sleeve always remains deflated around the bending section and thus maneuverability of the bending section is not compromised.

Furthermore, seeing that the sleeve remains always deflated around the probe, it almost does not increase the probe's outside diameter and therefore insertion of the endoscope within the patient's body can be carried out easier and without undue discomfort for a patient.

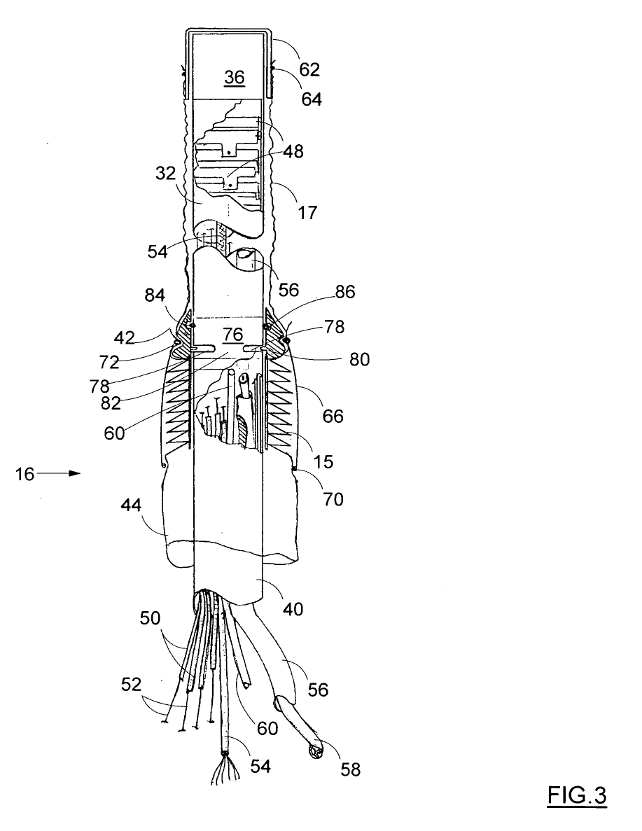

Referring to FIG. 3 it shows the enlarged portion of the distal section 16, which is shown schematically in a situation when the pressure is supplied to the sleeve and it begins to unfold and inflate. One can see that the inflated portion 44 of the sleeve surrounds the forwardmost region of the insertion tube 40. Various devices of the endoscope, which are necessary for its operation and functioning, are shown. Among those devices are e.g. vertebrae 48 of the bending section 32. The vertebrae are connected to cables 50 fitted with pull strings 52 for operating the vertebrae. Furthermore among those devices one can mention: a camera head 36 connected to an electric cable 54 for energizing the camera head, a guiding channel 56 with a working channel 58 insertable within the guiding channel, a tube 60 for supplying air as required for inflating the sleeve.

It is shown also that the forwardmost extremity of the sleeve terminates by a cap 62, to which the sleeve is sealingly attached for example by a spring seal 64. It is not shown specifically but should be understood that cap 62 is provided with a transparent window through which illuminating light can propagate so as the viewing optics of the camera head may observe the interior of the body passage.

An external skirt member 66 is provided, which surrounds an internal bushing member 68, put on the insertion tube. The folded portion of the sleeve is retained within an annular space provided between the external skirt member and the internal bushing member. The rear portion of the skirt member terminates by an annular shoulder 70, which causes the folded portion of the sleeve to unfold over the shoulder as soon as pressure is supplied to the sleeve through the air supply tube. By virtue of this provision, unfolding of the sleeve takes place in an organized fashion, i.e. evenly and without entanglement. The internal bushing and the skirt are configured as thin walled cylindrical bodies manufactured from plastic material, so as to impart flexibility to their walls and at the same time to retain their thickness at a minimum, which is required for retaining the sleeve and preventing its entanglement during unfolding. In practice the wall thickness of the skirt member and the bushing member is of about several tenths of a micron.

An O-ring 72, or any other connection means suitable for functioning as a seal spring, sealingly secures the frontal portion of the skirt member on a retaining ring 74. The retaining ring tightly seats on an intermediate ring 76. The retaining ring is coupled with possibility for detachment by a snap connection to the intermediate ring 76 put on the insertion tube behind the bending section. On the outward periphery of the intermediate ring are made at least two elongated grooves 78, extending transversally. The grooves are intended for snapping engagement with corresponding tongues 80 protruding from the inwardly facing periphery of the retaining ring and protruding towards the grooves.

At the end of the endoscopic procedure, the endoscope is evacuated from the body passage and is disconnected from the sleeve. This can be easily done if an operator manually twists the insertion tube together with the intermediate ring. By virtue of the transversal elongated grooves 78, relative rotational motion is allowed between the tongues and the grooves until the tongues escape from the grooves and are not any more in the snapping engagement and the insertion tube can be detached from the sleeve.

The intermediate ring is fitted with an inlet 82, which is deployed between the elongated grooves so as to be in flow communication with the air-supply tube 60.

The outwardly facing surface of retaining ring 74 is provided with an annular depression adapted for deployment of the O-ring 72. By virtue of this provision the skirt member is in sealing engagement with the retaining ring. Since the O-ring presses flexible sleeve 42 to the annular depression of the retaining ring, the space between the skirt member and the bushing member is sealed and therefore there is no propagation of air towards the forward portion 1 7 of the sleeve and it cannot inflate. Retaining ring 74 is fitted also with an annular groove 84, which accommodates therein a sealing ring 86, put over the intermediate ring 76. By virtue of this provision sealing arrangement is provided, which prevents propagating of air between retaining ring and the intermediate ring towards the forward portion 17, thus again preventing inflation of the forward portion of the sleeve.

It can be readily appreciated that the above described anchoring and sealing mechanism is very simple. This mechanism enables coupling the sleeve to the insertion tube with possibility for detachment and prevents the forward portion of the sleeve from inflation.

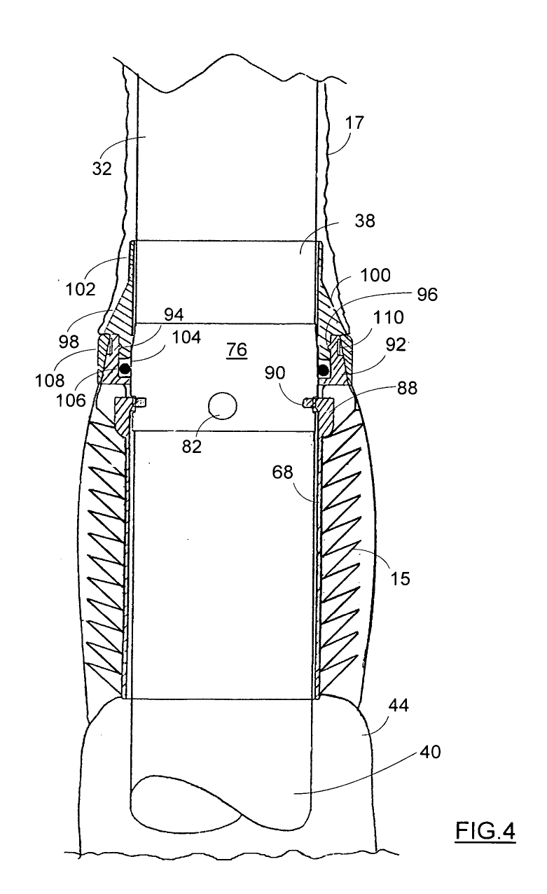

Now with reference to FIG. 4 an embodiment of the anchoring and sealing mechanism is shown. In this embodiment the elements which are common with the embodiment shown in FIG. 3 are designated by the same reference numerals. These elements include he frontal non-inflatable portion 17 of the sleeve, the probe section 32, the bending section 38, the insertion tube 40, the intermediate ring 76 with air inlet 82, the skirt member 66, the folded portion 15 of the sleeve, and the inflated portion 44 of the sleeve. It also comprises internal bushing member 68, however, in contrast to the previous embodiment, this bushing member is provided with a broadening tongue portion 88, which surrounds the intermediate ring 76. The tongue portion is fitted with tongues 90, which are adapted to be in snapping engagement with corresponding transversal grooves made on the outside periphery of the intermediate ring. By virtue of this provision the sleeve can be coupled to the intermediate ring with possibility for detachment.

The bushing member is provided with a forward annular shoulder 92, which is fitted with small protrusions 94 for engagement with a tail portion 96 of a retaining ring 98. This retaining ring similarly to the retaining ring of the previous embodiment is in sealing arrangement with the outside periphery of the intermediate ring. However in contrast with the previous embodiment the retaining ring is not provided with tongues for engagement with the intermediate ring. The outwardly facing periphery of the retaining ring is configured with a sloping section 100 and with a cylindrical section 102. By virtue of this, provision folded portion 15 of the sleeve smoothly converges with the non-inflatable portion 17.

The tail portion and the annular shoulder are configured and dimensioned in such a manner that an annular space 104 is provided therebetween. A sealing ring 106 is provided, which accommodates within this annular space and seals the unfolded portion of the sleeve from air, which could enter between the bushing member and the retaining ring.

A holding ring 108 is provided, which tightly fits the outside periphery of the shoulder 92 and presses it to the tail portion 98, thus securing the whole assembly together. The sleeve passes from the folded portion 15 to the non-inflatable portion 17 between the outwardly facing periphery of the shoulder 92 and the inwardly facing periphery of the holding ring 108.

The most forward extremity of the shoulder is fitted with an annular undercut 110 for accommodating thereinto a sealing ring (not shown) to seal the non-inflatable portion of the sleeve from air, which could enter between the shoulder and the holding ring.

Thus the anchoring and sealing arrangement of the embodiment shown in FIG. 4 allows coupling the sleeve to the insertion tube with possibility for detachment. At the same time this mechanism prevents the frontal portion of the sleeve from inflation and therefore the maneuverability of the endoscope's bending section is not compromised.

Similarly to the previous embodiment it is advantageous if the air inlet 82 is located between the grooves of the intermediate ring. It should be appreciated, however, that the inlet could be located in any other place, provided that its location prevents the air from approaching the forward portion of the sleeve.

It should be appreciated that the present invention is not limited to the above-described embodiments and that changes and one ordinarily skilled in the art can make modifications without deviation from the scope of the invention, as will be defined in the appended claims.

When used in the following claims, the meaning of terms “comprise”, “include”, “have” and their conjugates is “including but not limited to”.

It should also be appreciated that the features disclosed in the foregoing description, and/or in the following claims, and/or in the accompanying drawings may, both separately and in any combination thereof, are material for realizing the present invention in diverse forms thereof.

We claim:

1. An apparatus for endoscopic examination of a body passage, said apparatus including a flexible endoscope and an inflatable sleeve, said endoscope having a proximal end and a distal end, said distal end having an insertion tube, a bending section and a probe, wherein the improvement comprises said sleeve comprising a rear folded portion and a frontal unfolded portion, said rear portion of the sleeve being located behind the bending section, wherein, upon inflation of said sleeve, said rear portion of said sleeve unfolds and propels the endoscope within the body passage and said frontal portion of said sleeve remains unfolded.

2. The apparatus as defined in claim 1, in which said sleeve is coupled to the endoscope with a means for detachment.

3. The apparatus as defined in claim 2, in which said sleeve is coupled to the endoscope by an anchoring and sealing mechanism, wherein said mechanism attaches said sleeve to said endoscope at the beginning of an endoscopic examination and detaches said sleeve from said endoscope at the end of the endoscopic examination.

4. The apparatus as defined in claim 3, wherein said anchoring and sealing mechanism seals the frontal portion of the sleeve from inflation.

5. The apparatus as defined in claim 4, wherein said probe further comprises an optical head with a cap and viewing optics, wherein a forward most end of the frontal sleeve portion is attached to said cap.

6. The apparatus as defined in claim 5, wherein said cap includes a transparent window through which the viewing optics may observe an interior of the body passage.

7. The apparatus as defined in claim 6, wherein said insertion tube includes an internal bushing member and further comprising an external skirt member surrounding the bushing member to define an annular space between the bushing member and the skirt member, wherein said rear folded portion being retained within the annular space and unfolding therefrom.

8. The apparatus as defined in claim 7, wherein said skirt member is made of a plastic material.

9. The apparatus as defined in claim 7, wherein said anchoring and sealing mechanism further comprises an intermediate ring securable on the insertion tube behind the bending section, and further comprises a retaining ring detachably coupled with said intermediate ring, said skirt member being secured on the retaining ring.

10. The apparatus as defined in claim 9, wherein said skirt member being secured on the retaining ring by a seal spring.

11. The apparatus as defined in claim 9, wherein said retaining ring being coupled with the intermediate ring by a snap connection.

12. The apparatus as defined in claim 11, wherein said intermediate ring includes grooves and said retaining ring including corresponding tongues snappingly engageable with said grooves.

13. The apparatus as defined in claim 11, wherein said intermediate ring being provided with an inlet, which is in flow communication with an air supply tube.

14. The apparatus as defined in claim 13, wherein said inlet is situated between the grooves.

15. The apparatus as defined in claim 9, wherein said retaining ring being in sealing engagement with the intermediate ring.

16. The apparatus as defined in claim 7, wherein said anchoring and sealing mechanism further comprises a retaining ring and an intermediate ring, which is sealingly engageable with said retaining ring, wherein said retaining ring being detachably coupled with said skirt member.

17. The apparatus as defined in claim 16, wherein said intermediate ring includes grooves and said bushing member including corresponding tongues snappingly engageable with said grooves.

18. The apparatus as defined in claim 17, wherein said bushing member including a shoulder portion sealingly engageable with the retaining ring and the intermediate ring.

19. The apparatus as defined in claim 17, wherein said anchoring and sealing mechanism further comprises a holding ring sealingly engageable with the shoulder portion.

20. A method of propelling a probe of a flexible endoscope within a body passage, comprising providing said endoscope with an inflatable sleeve capable upon inflation to propel the probe within the body passage, wherein said sleeve comprises a rear folded portion and a frontal unfolded portion covering the probe, said method further comprises inflating the rear folded portion of the sleeve while keeping the frontal portion deflated.
